(19) United States
(12) Patent Application Publication
(10) Pub. No.: US 2025/0367453 A1
(43) Pub. Date: Dec. 4, 2025

(54) SYSTEMS AND METHODS FOR BIOMARKER-BASED PACING

(71) Applicant: BaroPace, Inc., Issaquah, WA (US)

(72) Inventor: Michael BURNAM, Issaquah, WA (US)

(73) Assignee: BaroPace, Inc., Issaquah, WA (US)

(21) Appl. No.: 19/134,051

(22) PCT Filed: Nov. 30, 2023
(86) PCT No.: PCT/US2023/081912

§ 371 Date: May 29, 2025

Related U.S. Application Data
(60) Provisional application No. 63/385,935, filed on Dec. 2, 2022.

(51) Int. Cl. A61N 1/365; A61N 1/02; A61N 1/362; G16H 10/60; G16H 40/67; G16H 50/20

(52) U.S. Cl. CPC A61N 1/36557; A61N 1/025; A61N 1/3629; A61N 1/36585; G16H 10/60; G16H 40/67; G16H 50/20

(57) ABSTRACT

Disclosed are methods, systems, and computer-readable medium for implementing a closed-loop system for determining an adjusted physiological pacing rate based on receiving one or more biomarker levels, comparing the one or more biomarker levels to a target biomarker level, and determining an adjusted physiological pacing rate based on comparing the one or more biomarker levels to the target biomarker level.

### CROSS-REFERENCE TO RELATED APPLICATIONS

This application claims priority to U.S. Provisional Application No. 63/385,935 filed Dec. 2, 2022, the entirety of which is incorporated by reference herein.

### TECHNICAL FIELD

Various embodiments of the present disclosure relate generally to cardiac pacing based on at least one biomarker.

### BACKGROUND

Physiological biomarkers, such as cortisol and Atrial Natriuretic Peptides (ANP), may be substances (e.g., hormones) secreted by the heart, brain, and/or other organs. Two types of these natriuretic peptides are b-type natriuretic peptide (BNP) (or brain natriuretic peptides) and N-terminal pro b-type natriuretic peptide (NT-proBNP). High levels of BNP may be indicative of heart failure, such as congestive heart failure. Stretching of cardiac tissue, as may occur due to a fluid-overloaded heart, stimulates atrial naturietic peptide release. For this reason, elevated atrial naturietic peptide levels (e.g. as measured as BNP) are accepted as a diagnostic marker of heart failure.

Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for calibrating a blood pressure measuring device.

In one aspect, an exemplary embodiment of a method for determining an adjusted physiologic pacing rate may include receiving one or more biomarker levels, wherein the biomarker levels indicate levels of at least one of atrial natriuretic peptide (ANP), B-type natriuretic peptide (BNP), N-terminal pro b-type natriuretic peptide (NT-proBNP), or C-type natriuretic peptide (CNP), comparing the one or more biomarker levels to a target biomarker level, and determining an adjusted physiologic pacing rate based on comparing the one or more biomarker levels to the target biomarker level.

In another aspect, an exemplary embodiment of a system for determining an adjusted physiologic pacing rate may include at least one memory storing instructions and at least one processor executing the instructions to perform a process. The at least one processor may be configured for receiving one or more biomarker levels, wherein the biomarker levels indicate levels of at least one of atrial natriuretic peptide (ANP), B-type natriuretic peptide (BNP), N-terminal pro b-type natriuretic peptide (NT-proBNP), or C-type natriuretic peptide (CNP), comparing the one or more biomarker levels to a target biomarker level, and determining an adjusted physiologic pacing rate based on comparing the one or more biomarker levels to the target biomarker level.

In a further aspect, an exemplary embodiment of a method for determining an adjusted physiologic pacing rate may include receiving one or more physiological inputs and one or more biomarker levels, wherein the biomarker levels includes levels of at least one of atrial natriuretic peptide (ANP), B-type natriuretic peptide (BNP), N-terminal pro b-type natriuretic peptide (NT-proBNP), or C-type natriuretic peptide (CNP), comparing the one or more biomarker levels to a target biomarker level, comparing the one or more physiological inputs to a threshold physiological input, determining a first adjusted physiologic pacing rate based on comparing the one or more biomarker levels to the target biomarker level, determining a second adjusted physiologic pacing rate based on comparing the one or more physiological inputs to the threshold physiological input, determining a first weight associated with the first adjusted physiologic pacing rate, determining a second weight associated with the second adjusted physiologic pacing rate, and determining an output adjusted physiologic pacing rate based on the first adjusted physiologic pacing rate, the first weight, the second adjusted physiologic pacing rate, and the second weight.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. Moreover, there are many embodiments described and illustrated herein.

FIG. 1 depicts an exemplary environment for determining an adjusted physiologic pacing rate, according to one or more embodiments.

FIG. 2 depicts a flowchart of an exemplary method for determining an adjusted physiologic pacing rate, according to one or more embodiments.

FIG. 3 depicts a further flowchart of an exemplary method for determining an adjusted physiologic pacing rate, according to one or more embodiments.

FIG. 4 depicts an example of training a machine learning model, according to one or more embodiments.

FIG. 5 depicts an example of a computing device, according to one or more embodiments.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION OF EMBODIMENTS

Various embodiments of the present disclosure relate generally to methods and systems for biomarker-based cardiac pacing.

According to implementations of the disclosed subject matter, physiological pacing may be determined based on physiological inputs such as blood pressure. Such physiological input based cardiac pacing may be used to treat conditions such as, but not limited to, drug resistant hypertension (DRH), DRH with diastolic congestive heart failure (DCHF), heart failure with preserved ejection fraction (HFpEF), etc.

Blood pressure may be detected using a blood pressure measuring device (a “device” or a “blood pressure device”). A blood pressure may be a sensed value, a blood pressure, a sensed value converted into one or more other formats (e.g., by a processor), or the like. A blood pressure may indicate how much pressure a user's blood exerts against the user's artery walls when the user's heart beats (e.g., a systolic blood pressure). A blood pressure may indicate how much pressure a user's blood exerts against the user's artery walls when the user's heart is resting between beats (e.g., diastolic blood pressure).

A blood pressure measuring device may include any type of blood pressure monitor or cuff such as, for example, a pneumatic cuff relying on mechanical compression of a peripheral artery cuff (e.g., to be attached to brachial artery, ankle, wrist, etc.), a non-pneumatic cuff (e.g., which analyzes an arterial waveform and function anywhere on the body where the arterial pulse contour can be sensed such as at a wrist), or an implantable sensor within a blood vessel or heart chamber. The blood pressure measuring device may be a light-based device such as a photoplethysmography (PPG) device.

Other physiological inputs include, but are not limited to, a biomarker level (e.g., cortisol, ANP, BNP, NT-proBNP, etc.), a blood oxygen level, glucose level, blood electrolytes level, a heart rate, an accelerometer value, a respiratory rate sensor value (e.g., via diaphragmatic movement), a thoracic impedance, an impedance (e.g., as a correlate of right ventricular function), an environmental parameter, an ambient oxygen concentration (e.g., SPO2), a humidity, portions of cardiac rate such as atrial rate, ventricular rate, atrioventricular conduction, the presence of rhythm irregularities, autonomic nervous system (ANS) function, glucose, skin electrolytes, galvanic skin response, PPG values, Electroencephalogram (EEG) wave, urination parameters, etc. Such physiological inputs may be provided by one or more sensors, devices, or the like. Systolic blood pressure (SBP) and diastolic blood pressure (DBP) may be sensed by one or more blood pressure sensing devices.

Accordingly, techniques disclosed herein may be implemented to modify pacing (e.g., by a pacing device such as a pacemaker) based on physiological inputs (e.g., blood pressure, biomarker levels, etc.). Pacing may be modified in accordance with an algorithm or machine learning output. For example, pacing may be modified to improve a blood pressure related condition by increasing or decreasing blood pressure based on observed biomarker levels. The modification may result an increase in a cardiac pacing rate or amplitude, a decrease in cardiac pacing rate or amplitude, an acceleration of a cardiac pacing rate, a deceleration of a cardiac pacing rate, and/or the like.

According to techniques disclosed herein, sub-threshold pacing and/or supra- threshold pacing (e.g., atrial pacing and/or ventricle pacing) may be used to treat heart conditions. For example, supra-threshold pacing may include providing electrical stimulation that meets a minimum threshold for pacing to cause a cardiac cycle. According to another example, sub-threshold pacing may include providing electrical stimulation that does not meet the minimum threshold for pacing to cause a cardiac cycle but may provide stimulation and/or release hormones (e.g., ATP, ANP, BNP, NT-proBNP, etc.) for therapeutic effect.

Such modified pacing may, at least in part, improve blood pressure based conditions for a patient. Such conditions may include hypertension, DRH, DRH with diastolic congestive heart failure (DCHF), HFpEF, and/or the like.

In an exemplary use case, atrial natriuretic peptide (ANP) activity may be determined using a B-type natriuretic peptide (BNP) (or brain natriuretic peptide) test or an N-terminal pro b-type natriuretic peptide (NT-proBNP) test which may be blood tests or other applicable tests to measure levels of BNP or NT-proBNP (BNP and NT-proBNP interchangeably referred to as BNP hereafter) hormones. BNP hormones may be created by or based on a body's response (e.g., a heart response) to heart conditions disclosed herein and may indicate ANP levels.

Blood levels of ANPs (e.g., as determined based on measured BNP levels) may be used as a marker of heart failure. When the heart is stretched from fluid overload in heart failure, ANPs in the blood may increase in concentration. Placing a pacemaker in patients with a heart conditions such as HFpEF may improve heart conditions (e.g., fewer hospitalizations, lower New York Heart Association (NYHA) classification, etc.).

According to implementations of the disclosed subject matter, contrary to conventional techniques, patients treated for heart conditions, in accordance with the techniques disclosed herein, may present an increase in ANP levels (e.g., as presented based on a measurement of BNP levels). According to implementations of the disclosed subject matter, increased ANP levels may be indicative of treatment of heart conditions based on the physiological input based cardiac pacing discussed herein.

For example, increased detected/measured BNP values (e.g., which can be a diagnostic indication of increased circulating ANP) may be indicative of a body's natural response to treat heart conditions. Treating such heart conditions using the physiological input based cardiac pacing discussed herein may also result in increased ANP hormone release. Such increased hormone activity may be determined by using a BNP test, as discussed herein. Accordingly, ANPs may be released by the electrical stimulation aspect of physiological input based on cardiac pacing, as disclosed herein.

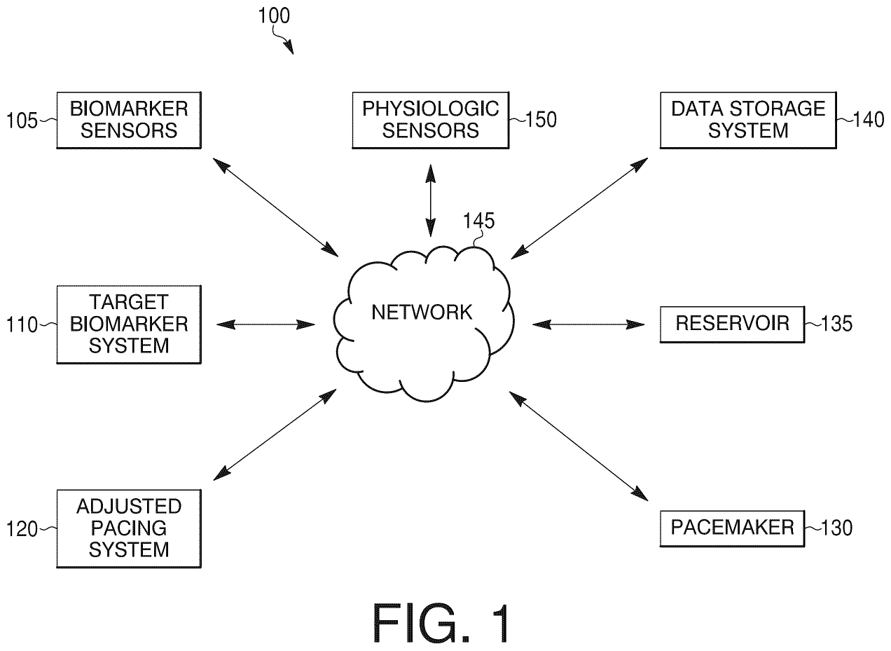

According to implementations of the disclosed subject matter, adjusted physiologic pacing rates (e.g., based on physiological input(s)) may be further determined based on detected ANP levels using a BNP test. For example, adjusted physiologic pacing rates (e.g., for atrial pacing) may be determined based on detected BNP levels in addition to, or independent of, determining adjusted physiologic pacing rates based on physiological inputs (e.g., blood pressure). Such adjusted physiologic pacing rates based on BNP levels and/or physiological inputs may result in treatment of heart conditions (e.g., heart failure). The treatment may be based on release of ANPs into a patient's blood stream. FIG. 1 shows an environment 100 for a closed-loop system determining physiological pacing rates based on detected/measured BNP levels and treating a heart condition via ANP hormones released as a result of pacing based on the physiological pacing rates. Such physiological pacing rate may be determined, for example, upon detection of a heart condition such as Hypertensive Heart Disease. Physiological pacing rates may include any applicable properties for cardiac pacing such as, but not limited to, frequency of pacing, amplitude of pacing, duration of pacing, acceleration of pacing, deceleration of pacing, etc.

While the examples above involve determining an adjusted physiologic pacing rate based on ANP/BNP levels, it should be understood that techniques according to this disclosure may be adapted to any suitable biomarker, e.g., a hormone, a protein, a peptide, cortisol, etc. As used herein, a biomarker may be any biological maker such as a substance, structure, or process that can be measured in the body or its products and may be used to influence or predict the incidence of an outcome or disease. A biomarker may be a characteristic that is objectively measured and evaluated as an indicator of biological processes, pathogenic processes, or pharmacologic responses to a therapeutic intervention. A biomarker may measure a biological, physiological, cellular, or molecular attribute. For example, a biomarker may be or may indicate a BNP level or presence, an ANP level or presence, a pulse, a chemical level or presence, a cortisol level or presence, a temperature, a protein level or presence, a vitamin level or presence, hemoglobin level or presence, testosterone level or presence, a triglyceride level or presence, a lipid level or presence, and/or the like. It should also be understood that the examples above are illustrative only. The techniques and technologies of this disclosure may be adapted to any suitable activity.

Presented below are various systems and methods of determining an adjusted physiologic pacing rate.

FIG. 1 depicts an exemplary environment for determining an adjusted physiologic pacing rate, according to one or more embodiments. Environment 100 of FIG. 1 depicts at least one biomarker sensor 105, a target biomarker system 110, an adjusted pacing system 120, a pacemaker 130, a reservoir 135, at least one physiological sensor 150, a data storage system 140, and a network 145.

At least one biomarker sensor 105 may be configured to determine levels of one or more biomarker. For example, at least one biomarker sensor 105 may be configured to determine the levels of ANPs, BNPs, C-type natriuretic peptide (CNPs), NT-proBNP, etc. In another example, at least one biomarker sensor 105 may be configured to determine the levels of cortisol. In some embodiments, at least one biomarker sensor 105 may be configured to utilize a rapid assay to determine levels of the one or more biomarker. In some embodiments, at least one biomarker sensor 105 may be configured to indirectly measure biomarker levels. For example, at least one biomarker sensor 105 may be configured to determine the biomarker levels based on systemic vascular resistance. At least one biomarker sensor 105 may be configured to receive data from other aspects of environment 100, e.g., target biomarker system 110, adjusted pacing system 120, pacemaker 130 (e.g., a processor of pacemaker 130), reservoir 135 (e.g., a processor of reservoir 135), at least one physiological sensor 150, and/or data storage system 140. At least one biomarker sensor 105 may be configured to transmit data to other aspects of environment 100, such as, to target biomarker system 110, adjusted pacing system 120, pacemaker 130 (e.g., a processor of pacemaker 130), reservoir 135 (e.g., a processor of reservoir 135), at least one physiological sensor 150, and/or data storage system 140.

Target biomarker system 110 may be configured to determine a target biomarker range and/or level. The target biomarker range and/or level may be determined based on user-specific and/or population-level data (e.g., city-, county-, state-, country-level data). For example, the target biomarker range and/or level may be determined based current and/or historical ANP levels from a given user. In another example, the target biomarker range and/or level may be determined based current and/or historical ANP levels from a given population. In a further example, the target biomarker range and/or level may be determined based on ANP levels from a given user as well as ANP levels from a given population. Target biomarker system 110 may be configured to receive data from other aspects of environment 100, e.g., at least one biomarker sensor 105, adjusted pacing system 120, pacemaker 130 (e.g., a processor of pacemaker 130), reservoir 135 (e.g., a processor of reservoir 135), at least one physiological sensor 150, and/or data storage system 140. Target biomarker system 110 may be configured to transmit data to other aspects of environment 100, e.g., target biomarker system 110, adjusted pacing system 120, pacemaker 130 (e.g., a processor of pacemaker 130), reservoir 135 (e.g., a processor of reservoir 135), at least one physiological sensor 150, and/or data storage system 140.

A user-specific target biomarker range and/or level may be determined based on at least one of a user's medical history, user medical characteristics (e.g., current or past medicines, medicine compliance, etc.), current and/or historical user physiological values (e.g., blood pressure values, biomarker values (e.g., cortisol, natriuretic peptides, etc.), etc. For example, a user's history of kidney failure may be utilized in determining the user-specific target biomarker range and/or level.

Adjusted pacing system 120 may be configured to determine an adjusted pacing rate based on levels of the one or more biomarker and the target biomarker range and/or level. As discussed in further detail below, adjusted pacing system 120 may be configured to determine the adjusted pacing rate via at least one of an algorithm, a trained machine learning model, a look-up table, an external system, etc. Adjusted pacing system 120 may be configured to receive data from other aspects of environment 100, e.g., at least one biomarker sensor 105, target biomarker system 110, pacemaker 130 (e.g., a processor of pacemaker 130), reservoir 135 (e.g., a processor of reservoir 135), at least one physiological sensor 150, and/or data storage system 140. Adjusted pacing system 120 may be configured to transmit data to other aspects of environment 100, such as to at least one biomarker sensor 105, target biomarker system 110, pacemaker 130 (e.g., a processor of pacemaker 130), reservoir 135 (e.g., a processor of reservoir 135), at least one physiological sensor 150, and/or data storage system 140.

Reservoir 135 may be configured to house at least one compound (e.g., a natriuretic peptide, a medication such as a blood pressure medication, a fluid, a drug, a solution, etc.). Reservoir 135 may be further configured to release the at least one compound. The compound to be released, a quantity of compound to be released, and/or a ratio or amounts of two or more compounds to be released may be based on the determined adjusted pacing rate or the data output by the one or more biomarker sensors 105. For example, if adjusted pacing system 120 determines that the required adjusted pacing rate would exceed a physiological threshold (e.g., a maximum pacing rate at which life may be sustained), adjusted pacing system 120 may transmit a request to reservoir 135 to release (e.g., into the bloodstream of the user) the at least one compound housed within reservoir 135.

One or more look-up tables, algorithms, and/or machine learning models disclosed herein may be used to output a combination of an adjusted physiological pacing rate and compound output from reservoir 135. According to implementations, a combination of an adjusted physiological pacing rate and compound output may be determined to cause an adjustment in a biomarker level, a physiological level, and/or the like. According to these implementations, the adjusted physiological pacing rate may be determined based at least in part on an expected effect of an amount or type of compound to be output. Similarly, the amount or type of compound to be output may be determined based at least in part on the expected effect of the adjusted physiological pacing rate.

Reservoir 135 may be configured to receive data from other aspects of environment 100, e.g., at least one biomarker sensor 105, target biomarker system 110, adjusted pacing system 120, pacemaker 130 (e.g., a processor of pacemaker 130), at least one physiological sensor 150, and/or data storage system 140. Reservoir 135 may be configured to transmit data to other aspects of environment 100, e.g., at least one biomarker sensor 105, target biomarker system 110, adjusted pacing system 120, pacemaker 130 (e.g., a processor of pacemaker 130), at least one physiological sensor 150, and/or data storage system 140.

At least one physiological sensor 150 may be configured to determine one or more physiological inputs, e.g., the levels and/or presence of one or more physiological inputs. For example, at least one physiological sensor 150 may be configured to determine the levels, measures, etc. of blood pressure, temperature, blood oxygen level, etc. In some embodiments, at least one physiological sensor 150 may use any suitable method (e.g., direct, indirect, assay, etc.) to determine the one or more physiological input. At least one physiological sensor 150 may be configured to receive data from other aspects of environment 100, e.g., at least one biomarker sensor 105, target biomarker system 110, adjusted pacing system 120, pacemaker 130 (e.g., a processor of pacemaker 130), reservoir 135 (e.g., a processor of reservoir 135), and/or data storage system 140. At least one physiological sensor 150 may be configured to transmit data to other aspects of environment 100, such as, to at least one biomarker sensor 105, target biomarker system 110, adjusted pacing system 120, pacemaker 130 (e.g., a processor of pacemaker 130), reservoir 135 (e.g., a processor of reservoir 135), and/or data storage system 140.

One or more of the components in FIG. 1 may communicate with each other and/or other systems, e.g., across network 145. In some embodiments, network 145 may connect one or more components of environment 100 via a wired connection. In some embodiments, network 145 may connect one or more aspects of environment 100 via an electronic network connection, for example a wide area network (WAN), a local area network (LAN), personal area network (PAN), or the like. In some embodiments, the electronic network connection includes the internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like. In any case, the connections within the environment 100 may be network, wired, any other suitable connection, or any combination thereof.

Although depicted as separate components in FIG. 1, it should be understood that a component or portion of a component in the environment 100 may, in some embodiments, be integrated with or incorporated into one or more other components. For example, target biomarker system 110 may be integrated in adjusted pacing system 120. In another example, pacemaker 130 may further include at least one biomarker sensor 105. In some embodiments, operations or aspects of one or more of the components discussed above may be distributed amongst one or more other components. In some embodiments, some of the components of environment 100 may be associated with a common entity, while others may be associated with a disparate entity. Any suitable arrangement and/or integration of the various systems and devices of the environment 100 may be used.

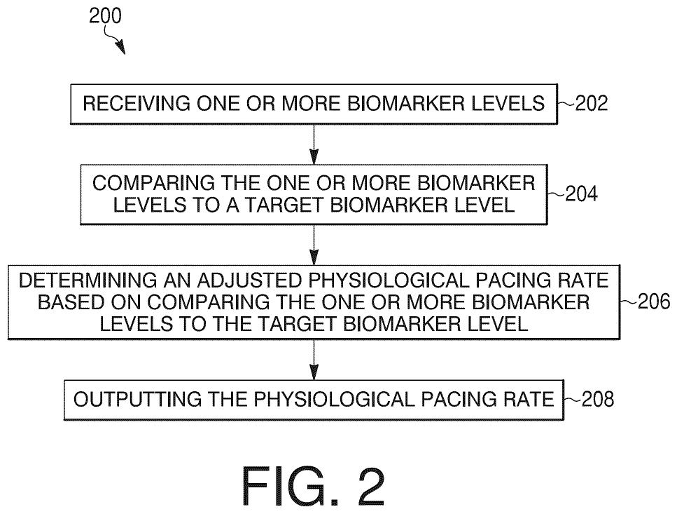

FIG. 2 depicts a method 200 for determining an adjusted physiologic pacing rate, according to one or more embodiments. At step 202, a patient's one or more biomarker levels may be received. The biomarker levels may be detected using a sensor and/or based on a biomarker test (e.g., an assay test). The biomarker levels may be provided to a processor that is local to a pacing device or a remote component external to the pacing device (e.g., a user device, a cloud component, an external processor, etc.). Alternatively, or in addition, physiological inputs (e.g., blood pressure) of a patient may be detected at step 202. The physiological inputs may be received from a physiological sensor, as described herein.

At step 204, the detected biomarker levels may be compared to a target biomarker level and/or one or more other factors, as disclosed herein. For example, detected biomarker levels may be in the range of approximately 0 to approximately 300, approximately 300 to approximately 450, or the like. For example, detected BNP levels that indicate heart failure (e.g., BNP levels over approximately 100 pg/ml may, NT-proBNP over approximately 125 pg/ml for patients under approximately 75 years old, NT-proBNP over approximately 450 pg/ml for patients over approximately 75 years old, NT-proBNP over approximately 125 pg/ml, etc.), may trigger step 204, 206, and/or 208 of method 200, as discussed herein. The target biomarker level may be a predetermined level, may be determined using an algorithm, or may be output by a machine learning model. The machine learning model may be trained to output target biomarker levels based on one or more of historical biomarker levels, historical target biomarker levels, patient medical history, patient medication history, patient demographic, patient vitals, or the like. For example, the machine learning model may output a target biomarker level for a given patient such that the target biomarker level for the given patient is different than the target biomarker level of a different patient.

The comparison at step 204 may include determining a magnitude of difference between a detected biomarker level and the target biomarker level. The magnitude may a value, a percent or ratio, a tier, or the like. According to implementations of the disclosed subject matter, a predictive formula, algorithm, or a machine learning model may be used to determine target biomarker levels based on one or more factors. Target biomarker levels may be based on, for example, one or more detected biomarker levels, a change in detected biomarker levels, a rate of change (e.g., increase or decrease) in detected biomarker levels, physiological inputs (e.g., blood pressure), patient symptoms, patient medications, patient history, or the like or a combination thereof. For example, a first detected biomarker level may be greater than 200 and a subsequent detected biomarker level may be a threshold amount (e.g., approximately 10%) lower than the first detected biomarker level. A target biomarker level may be determined based on the first level being greater than 200, the change in biomarker, or the like or a combination thereof.

As discussed herein, alternatively, or in addition, a target biomarker level may be determined based on one or more physiological inputs. For example, a target biomarker level may be determined based on a change in blood pressure (e.g., an elevated blood pressure that is elevated in comparison to a threshold blood pressure or one or more previous blood pressures).

A target biomarker level may be output by a machine learning model (e.g., a target biomarker level machine learning model). The machine learning model may be trained to output a target biomarker level based on one or more inputs. The machine learning model may be trained in accordance with techniques disclosed herein and may be trained based on historical or simulated patient biomarker levels, historical or simulated target patient biomarker levels, historical or simulated population (e.g., cohort) biomarker levels, and/or historical or simulated population target biomarker levels (e.g., training data). The machine learning model may be trained by modifying one or more layers, weights, biases, synapsis, and/or the like of the model based on the training data. The machine learning model may be trained to output a target biomarker level based on one or more inputs such as, but not limited to, a detected biomarker level (e.g., a BNP level, an ANP level, a cortisol level, a protein level, a chemical level, etc.), a change in a detected biomarker level (e.g., over a period of time or over the duration an event), a rate of change in detected biomarker levels, a rate of change (e.g., increase or decrease) in detected biomarker levels, one or more physiological inputs (e.g., blood pressure, temperature, sympathetic nerve activity (SNA), etc.), patient symptoms (e.g., based on subjective inputs or sensor data), patient medications (e.g., patient provided, received by a system or component, medication compliance information, etc.), patient history, cohort population information, or the like. Accordingly, the target biomarker level may be patient specific based on at least one or more inputs associated with the patient.

Similarly, one or more physiological level may be compared to a target physiological level in accordance with the techniques disclosed above in relation to biomarker levels and target biomarker levels. For example, at step 204, a target physiological level (e.g., a blood pressure level, a temperature, a blood oxygen level, etc.) may be determined in accordance with the techniques disclosed herein.

At step 206, an adjusted physiologic pacing rate may be determined at least in part based on the comparison and/or determination at step 204. For example, if the biomarker level is a given magnitude below the target biomarker level, then a pacing rate, frequency, amplitude, acceleration, and/or deceleration may be adjusted as a result of the given magnitude being below the target biomarker level. Similarly, if the biomarker level is a given magnitude above the target biomarker level, then a pacing rate, frequency, amplitude, acceleration, and/or deceleration may be adjusted as a result of the given magnitude being above the target biomarker level. It will be understood that the amount or degree of adjustment may vary based on the value, percent or ratio, or tier of the difference in magnitude between the detect biomarker level and the target biomarker level.

According to an implementation, at step 206, an adjusted physiological pacing rate (e.g., stimulus intensity) may be determined to cause a release of ANP. This physiological pacing rate and/or release of ANP may be independent of a current heart rate. For example, as discussed herein, the physiological pacing rate may correspond to supra-threshold pacing such that the physiological pacing rate causes a cardiac cycle and/or an adjustment to a cardiac cycle. Alternatively, or in addition, the physiological pacing rate may correspond to sub-threshold pacing such that heart tissue (e.g., atrial tissue) is stimulated based on the sub-threshold physiological pacing rate to cause ANP release. According to this implementation, a sub-threshold pacing based physiological pacing rate may have one or more properties (e.g., amplitude, frequency, etc.) that do not result in a paced beat or an adjusted paced beat but, rather, electrically stimulate a heart to cause a release of ANP, as a therapeutic measure. Such supra-threshold pacing and/or sub-threshold pacing may treat and/or mitigate a heart condition (e.g., Hypertensive Heart Disease) based on ANP release caused by the supra-threshold pacing and/or sub-threshold pacing at step 208. Accordingly, a heart condition may be treated or mitigated via a closed-loop system where detected ANP levels (e.g., using BNP values) and/or physiological inputs are used to determine physiological pacing rate, that cause release of ANP to treat the heart condition.

Similarly, the adjusted physiological pacing rate may further be based on one or more physiological levels, in accordance with the techniques disclosed above in relation to biomarker levels and target biomarker levels. For example, at step 206, a target physiological level (e.g., a blood pressure level, a temperature, a blood oxygen level, etc.) may be used to further determine an adjusted physiological pacing rate. As discussed herein, adjusting based on the target biomarker level and/or target physiological inputs may be weighted such that the adjusted physiological pacing rate is based on such weights. The weights may be determined based on one or more priority levels (e.g., where a given physiological input or biomarker level may be prioritized relatively higher than another given physiological input or biomarker level).

An adjusted physiological pacing rate may be output using a look-up table, an algorithm, and/or a machine learning model (e.g., an adjusted physiological pacing machine learning model). This machine learning model may be trained to output adjusted physiological pacing based on one or more inputs. This machine learning model may be trained in accordance with techniques disclosed herein and may be trained based on historical or simulated patient data, historical or simulated population (e.g., cohort) data, and/or the like. The machine learning model may be trained by modifying one or more layers, weights, biases, synapsis, and/or the like of the model based on the training data. The machine learning model may be trained to output an adjusted physiological pacing rate based on one or more inputs such as, but not limited to, a detected biomarker level (e.g., a BNP level, an ANP level, a cortisol level, a protein level, a chemical level, etc.), a target biomarker level, a change in a detected biomarker level (e.g., over a period of time, over the duration an event, a historical patient change value), a rate of change in detected biomarker levels (e.g., a historical patient rate of change), a rate of change (e.g., increase or decrease) in detected biomarker levels, one or more physiological inputs (e.g., blood pressure, temperature, sympathetic nerve activity (SNA), etc.), patient symptoms (e.g., based on subjective inputs or sensor data), patient medications (e.g., patient provided, received by a system or component, medication compliance information, etc.), patient history, cohort population information, or the like. Accordingly, the adjusted physiological pacing rate may be patient specific based on at least one or more inputs associated with the patient.

According to implementations of the disclosed subject matter, the adjusted physiological pacing rate may be based on two or more target values. The two or more target values may include, for example, one or more target biomarker levels, one or more physiological outputs, one or more pacing limitations, and/or the like. For example, the adjusted physiological pacing rate may be determined based on the target biomarker level and a target blood pressure. For example, adjusted physiological pacing machine learning model may receive, as inputs, at least a detected cortisol biomarker level and a detected blood pressure level. The adjusted physiological pacing machine learning model may determine an adjusted physiological pacing rate based on both a target cortisol biomarker level and a target blood pressure level. According to this implementation, the adjusted physiological pacing rate may weight each target factor (e.g., target cortisol biomarker level and a target blood pressure level). The weight may be determined by the machine learning model based on historical data and/or preferred (e.g., predetermined) priorities. For example, the adjusted physiological pacing rate may be based on a target blood pressure adjustment that is weight higher than a target cortisol level adjustment such that the target blood pressure adjustment is prioritized over the target cortisol level adjustment, in accordance with the corresponding weights.

For example, the adjusted physiological pacing rate machine learning model may apply a weight of approximately 0.8 to any target blood pressure based adjustment and a weight of approximately 0.5 to any target cortisol level based adjustment. Accordingly, an optimal physiological pacing rate adjustment based on the target blood pressure may be adjusted (e.g., multiple) by approximately 0.8 and an optimal physiological pacing rate adjustment based on the target cortisol pressure may be adjusted (e.g., multiple) by approximately 0.5. The resulting adjustment may be normalized (e.g., averaged) to determine the adjusted physiological pacing rate.

According to implementations disclosed herein, the adjusted physiological pacing rate machine learning model may output a first physiological pacing rate based on a first target (e.g., target blood pressure level) at a first frequency. Similarly, the adjusted physiological pacing rate machine learning model may output a second physiological pacing rate based on a second target (e.g., target BNP level) at a second frequency. It will be understood that the first or second frequencies may be different frequencies and that the given adjusted pacing rate (e.g., based on the first frequency) may be adjusted (e.g., fine-tuned) at the second frequency. It will also be understood that if a first relatively higher weighted adjustment (e.g., based on blood pressure) is counter to a second a second relatively lower weighted adjustment (e.g., based on BNP levels), then only the first relatively higher weighted adjustment may be output. For example, if the adjusted physiological pacing rate based on a first relatively higher weighted adjustment (e.g., based on blood pressure) requires an increased pacing rate and if the adjusted physiological pacing rate based on a second relatively lower weighted adjustment (e.g., based on BNP levels) requires a decreased pacing rate, an increased pacing rate may be output based only on the first relatively higher weighted adjustment. Alternatively, an increased pacing rate may be based on both the first relatively higher weighted adjustment and the second relatively lower weighted adjustment (e.g., such that the increase in pacing rate is lower than if based only on the first relatively higher weighted adjustment).

It will be understood that an adjusted physiologic pacing rate may be determined and/or provided in response to a detected biomarker level, a medical diagnosis (e.g., a heart failure diagnosis), a medical condition determination (e.g., heart failure determination) based on a physiological input, or the like (e.g., based on a heart rate, based on a BNP level, etc.). It will be understood that an adjusted physiologic pacing rate may further be adjusted based on a physiological input (e.g., blood pressure, as discussed herein). For example, a blood pressure value for the patient may be detected and may be compared to a target blood pressure value. Accordingly, the adjusted physiological pacing rate may be determined based both on the comparison between the detected BNP and target BNP at step 206 and may further be determined based on the comparison between the detected blood pressure and the target blood pressure.

At step 208, the adjusted physiologic pacing rate may be output and the output may be received at a cardiac pacing device. The cardiac pacing device may be configured to pace based on the adjusted physiological pacing rate. Accordingly, the disclosed subject matter provides a closed-loop system for determining physiological pacing rates (e.g., based on ANP hormones or BNP values, based on physiological inputs such as blood pressure, etc.), stimulating a heart based on the physiological pacing rates (e.g., stimulating atrial tissue), and causing ANP release to treat a heart condition. The closed-loop system steps may be iterated such that after implementation of a physiological pacing rate and a corresponding ANP release, a new physiological pacing rate (e.g., based on updated ANP hormones or BNP values, based on updated physiological inputs such as blood pressure, etc.), is determined and may cause further ANP release to treat the heart condition.

As disclosed, one or more BNP levels may be sensed at step 202. A decision may be made whether to change a physiological pacing rate based on, for example, the one or more BNP levels, a change in detected BNP levels, a rate of change (e.g., increase or decrease) in detected BNP levels, physiological inputs (e.g., blood pressure), patient symptoms, patient medications, patient history, or the like or a combination thereof. Based on the determination, a target BNP level may be determined. At step 204, the one or more BNP levels sensed at step 202 may be compared to the target BNP level. At step 206, adjusted physiological pacing rates may be determined based on the comparison at step 204. Steps 202-206 may be repeated continuously, at given time intervals, and/or based on a trigger action. The trigger action may be, for example, a user input, a physiological input change, an expiration of a time period, and/or the like. An adjusted physiological pacing rate may be further refined by a machine learning model output. The machine learning output may be based on inputs provided to a machine learning model that include one or more of the one or more BNP levels, a change in detected BNP levels, a rate of change (e.g., increase or decrease) in detected BNP levels, physiological inputs (e.g., blood pressure), patient symptoms, patient medications, patient history, or the like. A resulting adjusted physiological pacing rate may be determined based on the machine learning output and, at 208, the adjusted physiological pacing rate may be output (e.g., to a pacing device).

According to an implementation, an adjusted physiological pacing rate may releases ANPs, which may remain above a threshold amount (e.g., detected as BNP levels) while the adjusted physiological pacing rate is applied. When the adjusted physiological pacing rate is no longer applied (e.g., when a patient is clinically improved, when a heart failure condition is not detected, etc. the ANP (measured as BNP levels) may decrease or remain within a range (e.g., within an expected ANP/BNP level range). If BNP levels above an expected range are detected after the adjusted physiological pacing rate is no longer applied, then steps 202-208 of method 200 may be repeated as disclose herein.

According to an implementation of the disclosed subject matter, BNP levels may be tested using an assay test, an antibody test, a blood test or the like. The comparison at step 204 using a target BNP level may be based on the type of test used to determine a BNP value. For example, the target BNP based on an assay test may be different than the target BNP based on an antibody test.

According to the implementations disclosed herein, at least one biomarker sensor 105 may detect one or more biomarkers on a continuous or discrete basis. For example, at least one biomarker sensor 105 may continuously measure a biomarker and may output a sensor reading corresponding to the continuous measurement of the respective biomarker. A processor may receive the continuous measurement and may trigger a trigger action, as further discussed herein. The trigger action may be based on a continuous or discrete biomarker level (e.g., based on a biomarker reading) exceeding a threshold level and/or exceeding a rate of change greater than a threshold rate of change of the corresponding biomarker level.

As another example of the implementations disclosed herein, at least one biomarker sensor 105 may detect cortisol levels. The at least one biomarker sensor 105 may include a salivary sensor that detects cortisol levels using a salivary sample. Alternatively, or in addition the at least one biomarker sensor 105 may include a pituitary sensor or adrenal gland sensor that detects a biomarker that corresponds to cortisol levels. The output of the at least one biomarker sensor 105 may be used determine cortisol levels. For example, a biomarker level, reading, or sample associated with the salivary sample, based on pituitary sensor outputs, and/or based on adrenal gland outputs may undergo a rapid assay analysis. The rapid assay analysis may occur using the at least one biomarker sensor 105 and/or a component in connection and/or communication with the at least one biomarker sensor 105. The output of such rapid assay analysis may be used to determine the cortisol levels. It will be understood a rapid assay analysis, as discussed above, may be used to analyze any biomarker sensor 105 reading or output to determine a corresponding physiological, biological, molecular, and/or cellular level or presence.

The determined cortisol level may be compared to a target biomarker (e.g., cortisol) level. As discussed herein, the target biomarker level may be based on a given patient (e.g., based on the patient's historical biomarker levels) and/or based on cortisol levels of a given population. A machine learning model may be trained to output a target biomarker level. The machine learning model may be trained in accordance with techniques disclosed herein and may be trained based on historical or simulated patient biomarker levels, historical or simulated target patient biomarker levels, historical or simulated population (e.g., cohort) biomarker levels, and/or historical or simulated population target biomarker levels (e.g., training data). The machine learning model may be trained by modifying one or more layers, weights, biases, synapsis, and/or the like of the model based on the training data.

An adjusted physiological pacing rate may be determined based on comparing a detected cortisol level to the target biomarker (e.g., cortisol) level. The adjusted physiological pacing rate may be determined such that output of the adjusted rate is expected to cause the detected cortisol level to reach the target biomarker (e.g., cortisol) level or to cause the detected cortisol level to reach a value between the detected cortisol level and the target biomarker (e.g., cortisol) level.

According to an implementation, an adjusted physiological pacing rate may be determined based on the likelihood of change score. The likelihood of change score may correspond to the likelihood that an output of the adjusted physiological pacing rate will cause the detected biomarker level to reach the target biomarker level or the likelihood that an output of the adjusted physiological pacing rate will cause the detected cortisol level to reach a value or range of values between the detected cortisol level and the target biomarker (e.g., cortisol) level. The machine learning model used to output the adjusted physiological pacing rate may determine the likelihood of change score and/or may generate the output adjusted physiological pacing rate based on an input likelihood of change score. The adjusted physiological pacing rate may be output such that a pacing device is configured to implement the adjusted physiological pacing rate for a given patient.

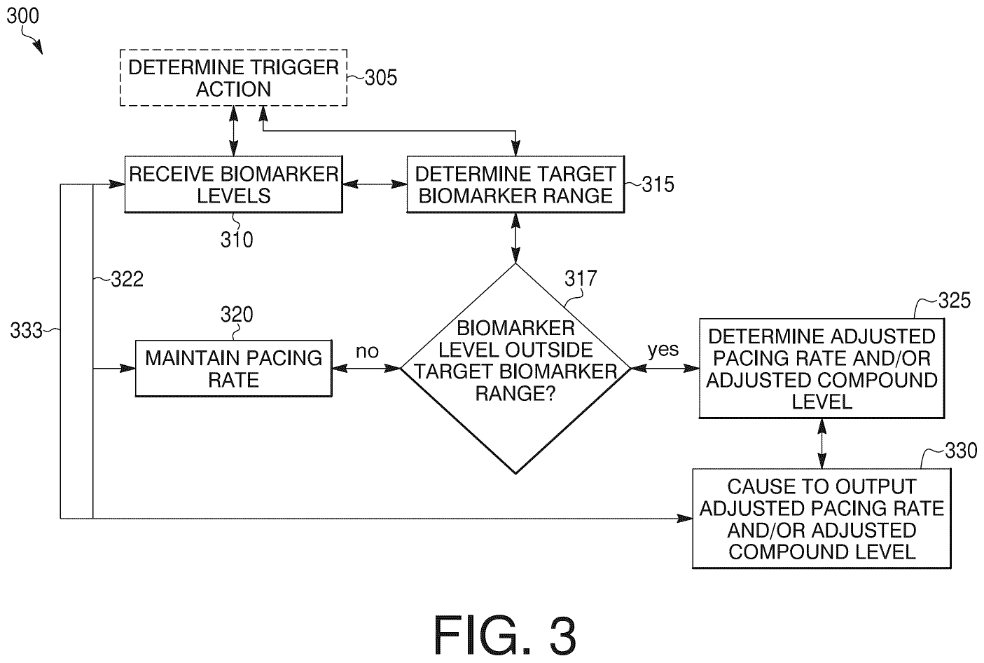

FIG. 3 depicts a method 300 for determining an adjusted physiologic pacing rate, according to one or more embodiments. Method 300 may include any of the steps discussed in relation to method 200, unless provided otherwise herein. Optionally, at step 305, a trigger action may be determined as discussed above, e.g., via at least one of at least one biomarker sensor 105, target biomarker system 110, adjusted pacing system 120, pacemaker 130, etc. In some embodiments, the determination of the trigger action at step 305 may result in the generation of the request for biomarker levels (step 310) and/or a determination of a target biomarker range (step 315). At step 310, biomarker levels may be received, e.g., from at least one biomarker sensor 105. The biomarker levels may be determined in response to a request, e.g., based on the determination of a trigger action at step 305. The biomarker levels may be determined as discussed herein, e.g., by target biomarker system 110, adjusted pacing system 120, etc.

At step 315, a target biomarker range may be determined, e.g., by target biomarker system 110. The target biomarker range may be determined using one or more techniques described herein.

At step 317, the detected biomarker levels may be compared to a target biomarker level and/or one or more other factors, as discussed in relation to step 204 in FIG. 2. For example, the detected biomarker levels may be compared to a target biomarker level and a patient medical record to determine whether the detected biomarker level is outside (e.g., is above or below) the target biomarker range. If the detected biomarker level is determined within the target biomarker range, the pacing rate may be maintained (see step 320). In some embodiments, in response to a determination that the pacing rate should be maintained, steps 305-317 may be repeated (see step 320).

If the detected biomarker level is determined to be outside the target biomarker range, an adjusted pacing rate and/or an adjusted compound level may be determined at step 325. For example, the pacing rate may be adjusted to increase or decrease based on the determination at step 317. In a further example, the compound level (e.g., the compound or amount of compound that may be released from reservoir 135) may be increased or decreased based on the determination at step 317.

At step 330, the adjusted pacing rate and/or the adjusted compound level may be caused to be output, e.g., by adjusted pacing system 120 and/or reservoir 135 (e.g., the processor associated with reservoir 135), respectively. In some embodiments, in response to a determination that the output adjusted pacing rate and/or adjusted compound level, steps 305-325 may be repeated (see step 333).

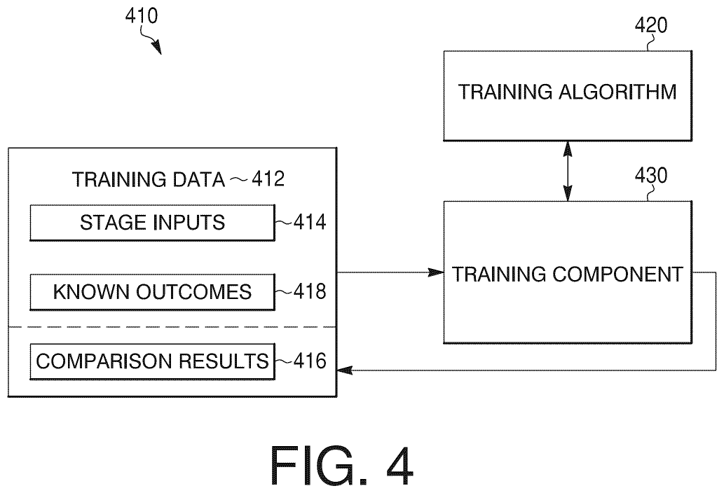

One or more implementations disclosed herein may be applied by using a machine learning model, another AI system such as a neural network, or a non-AI rules-based system. For example, a machine learning model may be used to determine a state machine and/or a next state. As shown in flow diagram 410 of FIG. 4, training data 412 may include one or more of stage inputs 414 and known outcomes 418 related to a machine learning model to be trained. The stage inputs 414 may be from any applicable source including an input or system discussed herein (e.g., an output from a step of FIGS. 1-3). The known outcomes 418 may be included for machine learning models generated based on supervised or semi-supervised training. An unsupervised machine learning model might not be trained using known outcomes 418. Known outcomes 418 may include known or desired outputs for future inputs similar to or in the same category as stage inputs 414 that do not have corresponding known outputs.

The training data 412 and a training algorithm 420 may be provided to a training component 430 that may apply the training data 412 to the training algorithm 420 to generate a machine learning model. According to an implementation, the training component 430 may be provided comparison results 416 that compare a previous output of the corresponding machine learning model to apply the previous result to re-train the machine learning model. The comparison results 416 may be used by the training component 430 to update the corresponding machine learning model. The training algorithm 420 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as the flows and/or process discussed herein (e.g., in FIGS. 1-4), etc., may be performed by one or more processors of a computer system, such any systems or devices used to implement the techniques disclosed herein. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

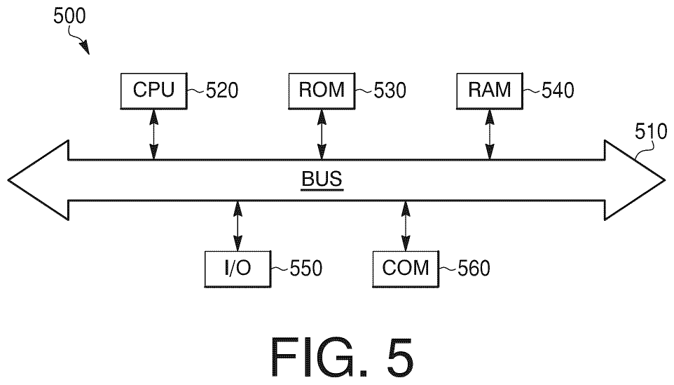

FIG. 5 depicts an example system 500 that may execute techniques presented herein. FIG. 5 is a simplified functional block diagram of a computer that may be configured to execute techniques described herein, according to exemplary embodiments of the present disclosure. Specifically, the computer (or “platform” as it may not be a single physical computer infrastructure) may include a data communication interface 560 for packet data communication. The platform may also include a central processing unit (“CPU”) 520, in the form of one or more processors, for executing program instructions. The platform may include an internal communication bus 510, and the platform may also include a program storage and/or a data storage for various data files to be processed and/or communicated by the platform such as ROM 530 and RAM 540, although the system 500 may receive programming and data via network communications. The system 500 also may include input and output ports 550 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

As discussed herein, a memory may include a device or system that is used to store information for immediate use in a computer or related computer hardware and digital electronic devices. Contents of memory can be transferred to storage (e.g., via virtual memory). Memory may be implemented as semiconductor memory, where data is stored within memory cells built from MOS transistors on an integrated circuit. Semiconductor memory may include volatile and/or non-volatile memory. Examples of non-volatile memory include flash memory and read-only memory (ROM), programmable read-only memory (PROM), erasable programmable read-only memory (EPROM), electrically erasable programmable read-only memory (EEPROM) memory, and the like. Examples of volatile memory include primary memory such as dynamic random-access memory (DRAM) and fast CPU cache memory such as static random-access memory (SRAM).

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A method for determining an adjusted physiologic pacing rate, the method comprising:
receiving one or more biomarker levels, wherein the biomarker levels indicate levels of at least one of atrial natriuretic peptide (ANP), B-type natriuretic peptide (BNP), N-terminal pro b-type natriuretic peptide (NT-proBNP), or C-type natriuretic peptide (CNP);
comparing the one or more biomarker levels to a target biomarker level; and
determining an adjusted physiologic pacing rate based on comparing the one or more biomarker levels to the target biomarker level.

2. The method of claim 1, further comprising determining of at least one of the ANP, the BNP, the NT-proBNP, or the CNP based on the one or more biomarker level.

3. The method of claim 1, further comprising:
receiving one or more physiological inputs;
comparing the one or more physiological inputs to a threshold physiological input; and
determining an adjusted physiologic pacing rate further based on comparing the one or more physiological inputs to the threshold physiological input.

4. The method of claim 1, further comprising outputting the adjusted physiological pacing rate.

5. The method of claim 1, further comprising causing a pacing device to pace based on the adjusted physiological pacing rate.

6. The method of claim 1, further comprising causing a compound reservoir to release a compound based on one or both of the one or more biomarker level or the determined adjusted physiologic pacing rate.

7. The method of any of claim 1, wherein the adjusted physiologic pacing rate is output by a trained machine learning model.

8. The method of claim 7, wherein the trained machine learning model is trained based on one or more of historical biomarker levels, historical target biomarker levels, a patient medical history, a patient medication history, a patient demographic, or patient vitals.

9. The method of claim 1, wherein the adjusted physiological pacing rate is further determined based on a magnitude of difference between the biomarker level and the target biomarker level.

10. The method of claim 1, further comprising:
determining the target biomarker level based on one or more of the one or more biomarker levels, a change in the one or more biomarker levels, a rate of change in the one or more biomarker levels, one or more physiological inputs, one or more patient symptoms, one or more patient medications, a patient history.

11. The method of claim 1, wherein the adjusted physiological pacing rate is one of a sub-threshold pacing rate or a supra-threshold pacing rate.

12. The method of claim 1, wherein the adjusted physiological pacing rate causes stimulation of a heart, wherein the stimulation of the heart causes a release of hormones.

13. The method of claim 12, wherein the released hormones treat a heart condition.

14. A system for determining an adjusted physiologic pacing rate, the system comprising:
at least one memory storing instructions; and
at least one processor executing the instructions to perform a process, the at least one processor configured to:
receiving one or more biomarker levels, wherein the biomarker levels indicate levels of at least one of atrial natriuretic peptide (ANP), B-type natriuretic peptide (BNP), N-terminal pro b-type natriuretic peptide (NT-proBNP), or C-type natriuretic peptide (CNP);
comparing the one or more biomarker levels to a target biomarker level; and
determining an adjusted physiologic pacing rate based on comparing the one or more biomarker levels to the target biomarker level.

15. The system of claim 14, the process further comprising:
receiving one or more physiological inputs;
comparing the one or more physiological inputs to a threshold physiological input; and
determining an adjusted physiologic pacing rate further based on comparing the one or more physiological inputs to the threshold physiological input.

16. The system of claim 14, wherein the adjusted physiologic pacing rate is determined via a trained machine learning model that has been trained based on one or more of historical biomarker levels, historical target biomarker levels, a patient medical history, a patient medication history, a patient demographic, or patient vitals.

17. The system of claim 14, wherein the adjusted physiological pacing rate is further determined based on a magnitude of difference between the biomarker level and the target biomarker level.

18. The system of claim 14, the process further comprising:
determining the target biomarker level based on one or more of the one or more biomarker levels, a change in the one or more biomarker levels, a rate of change in the one or more biomarker levels, one or more physiological inputs, one or more patient symptoms, one or more patient medications, a patient history.

19. A method for determining an adjusted physiologic pacing rate, the method comprising:
receiving one or more physiological inputs and one or more biomarker levels, wherein the biomarker levels includes levels of at least one of atrial natriuretic peptide (ANP), B-type natriuretic peptide (BNP), N-terminal pro b-type natriuretic peptide (NT-proBNP), or C-type natriuretic peptide (CNP);
comparing the one or more biomarker levels to a target biomarker level;
comparing the one or more physiological inputs to a threshold physiological input;
determining a first adjusted physiologic pacing rate based on comparing the one or more biomarker levels to the target biomarker level;
determining a second adjusted physiologic pacing rate based on comparing the one or more physiological inputs to the threshold physiological input;
determining a first weight associated with the first adjusted physiologic pacing rate;
determining a second weight associated with the second adjusted physiologic pacing rate; and
determining an output adjusted physiologic pacing rate based on the first adjusted physiologic pacing rate, the first weight, the second adjusted physiologic pacing rate, and the second weight.

20. The method of claim 19, wherein the first weight or the second weight is based on a predetermined priority level.
